(19) United States
(12) Patent Application Publication
(10) Pub. No.: US 2020/0069231 A1
(43) Pub. Date: Mar. 5, 2020

(54) EEG-BASED PSYCHOLOGICAL TEST METHOD AND TERMINAL DEVICE

(71) Applicant: Neural FLEX Technology (Shenzhen) CO., LTD, Shenzhen (CN)

(72) Inventors: Chao Feng, Shenzhen (CN); Xianhua Li, Shenzhen (CN); Wenming Yi, Shenzhen (CN); Tao Gong, Shenzhen (CN); Ding Yuan, Shenzhen (CN)

(21) Appl. No.: 16/371,138

(22) Filed: Apr. 1, 2019

(30) Foreign Application Priority Data
Aug. 31, 2018 (CN) 201811015387.2

(51) Int. Cl. A61B 5/16; G06T 7/00; G06K 9/62; A61B 5/0484; A61B 5/00

(52) U.S. Cl. CPC A61B 5/165; G06T 7/0012; G06K 9/6257; G06T 2207/20081; G06K 9/6259; A61B 5/04842; A61B 5/6803; G06K 9/6267

(57) ABSTRACT

The present disclosure relates to the field of computer technology, and provides an EEG-based psychological test method and a terminal device. The method includes: collecting, by an EEG acquisition device, an EEG signal of a subject when test information is felt by the subject, inputting the test information into a machine learning-based information recognition model, and obtaining a recognition result output from the information recognition model; and determining a psychological test result according to the EEG signal and the recognition result of the test information corresponding to the EEG signal in time.

### CROSS-REFERENCES TO RELATED APPLICATION

This application claims priority to Chinese Patent Application 201811015387.2, filed on Aug. 31, 2018, the content of which is incorporated herein by reference in its entirety.

### TECHNICAL FIELD

The present disclosure relates to the field of computer technology, and in particular, to an EEG-based psychological test method and a terminal device.

### BACKGROUND

A principle of applying brainwaves for psychological test is that when humans see stimuli, the response to the stimuli will be reflected in the brainwave signal, and these changes cannot be easily controlled by the test taker. For ordinary people, relevant tests are helpful to analyze the mental state or thinking patterns of people, such as the source of stimulation of psychological changes. For drug abusers under detoxification, the relevant test may be used to test whether the psychological detoxification is successful. For the polygraph detector, the relevant test may be used to check if he or she is lying.

In the process of testing the mental state using brain waves, it is necessary to use a predetermined input data set. For example, it is necessary to use a pre-edited image, text, sound and other stimulus sequences; for the drug addict, it is necessary to use a questionnaire to investigate the relapse stimulus source of the drug addict, and then input the relevant stimulation information into the system. The editing of the input data set is time-consuming and labor-intensive, and the predetermined input data set coverage is fixed, which limits the applicable scenarios of the test.

### SUMMARY

In view of this, embodiments of the present disclosure provide an EEG-based psychological test method and a terminal device, so as to solve the problem that the test applicable scenario is limited because in the current EEG-based psychological test process a predetermined input data set is a required, and the coverage of the input data set is fixed.

According to a first aspect of the present disclosure, it is provided an EEG-based psychological test method including:

collecting, by an EEG acquisition device, an EEG signal of a subject when test information is felt by the subject;

inputting the test information into a machine learning-based information recognition model, and obtaining a recognition result output from the information recognition model; and

determining a psychological test result according to the EEG signal and the recognition result of the test information corresponding to the EEG signal in time.

According to a second aspect of the present disclosure, it is provided a terminal device including a memory, a processor, and a computer program stored in the memory and operable in the processor, where the processor is configured to execute the computer program to implement steps of the method of the first aspect.

According to a third aspect of the present disclosure, it is provided a computer readable storage medium with a computer program stored therein, wherein when the computer program is executed by a processor, steps of the method of the first aspect.

Compared with the prior art, the embodiment of the present disclosure has the beneficial effects as follows. By automatically recognizing the test information by using a machine learning-based information recognition model, and then determining the psychological test result according to the EEG signal of the subject and the recognition result of the test information corresponding to the EEG signal in time, the test information can be recognized by using the information recognition model and there is no need to edit and collate the test information, thereby the test process can be simplified, the test efficiency can be improved, and the adoption range and data amount of the test information can be enlarged. Thus, the applicability of the psychological test scenario can be enhanced, the test result can be improved and psychological test can be made more convenient and practical.

### BRIEF DESCRIPTION OF THE DRAWINGS

In order to more clearly illustrate the technical solutions in the embodiments of the present disclosure, the drawings used in the description to the embodiments or the prior art will be briefly described below. It is obvious that the drawings in the following description are just for some embodiments of the present disclosure, those skilled in the art can also obtain other drawings based on these drawings without paying any creative effort.

FIG. 1 is a flowchart of an implementation of an EEG-based psychological test method according to an embodiment of the present disclosure;

FIG. 2 is a flowchart showing an implementation of recognizing a special EEG signal in an EEG-based psychological test method according to an embodiment of the present disclosure;

FIG. 3 is a schematic diagram of an EEG acquisition device according to an embodiment of the present disclosure;

FIG. 4 is a block diagram of an implementation of an implementation of an EEG-based psychological test method according to an embodiment of the present disclosure;

FIG. 5 is a schematic diagram of an EEG-based psychological test apparatus according to an embodiment of the present disclosure; and

FIG. 6 is a schematic diagram of a terminal device according to an embodiment of the present disclosure.

### DESCRIPTION OF THE EMBODIMENTS

In the following description, in order to describe but not intended to limit, concrete details such as specific system structure, technique, and so on are proposed, thereby facilitating comprehensive understanding of the embodiments of the present application. However, it will be apparent to the ordinarily skilled one in the art that, the present application can also be implemented in some other embodiments without these concrete details. In some other conditions, detailed explanations of method, circuit, device and system well known to the public are omitted, so that unnecessary details can be prevented from obstructing the description of the present application.

In order to explain the technical solutions described in the present disclosure, the following description will be made by way of specific embodiments.

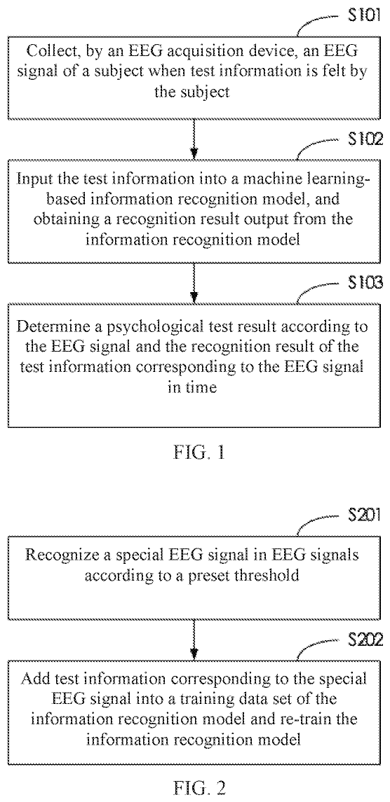

FIG. 1 is an implementation of an EEG-based psychological test method according to an embodiment of the present disclosure. The method is described in detail as follows.

In S101, an EEG signal of a subject is collected by an EEG acquisition device when test information is felt by the subject.

In this embodiment, the execution entity may be a terminal device, and the terminal device may be a computing device such as a desktop computer, a laptop computer, a palmtop computer, a mobile phone, a server or the like, which is not limited herein. The EEG acquisition device is a device for collecting an EEG signal of a subject, and may be, for example, a head-mounted EEG acquisition device or other forms of acquisition device, which is not limited herein. The terminal device can obtain the EEG signal collected by the EEG acquisition device.

The test information may include, but is not limited to, one or more of a picture, an audio, a video, and a text, which are not limited herein. For example, the test information may be a picture collection composed of multiple pictures. The terminal device may play the test information by itself or through other playing devices to present the test information to the subject, so as to acquire the EEG signals when the subject feels different test information. The subject is a person who needs to perform a psychological test, the subject feeling the test information may be the subject visually watching the video or picture, or the subject listening to the audio by hearing, or the subject experiencing other forms of test information through other means of perception, etc., which are not limited herein.

Optionally, the EEG signal comprises at least one selected from a group consisting of an event-related potential signal, a self-generating potential signal, a motion-imagery EEG signal and a visual-evoked potential signal.

Among them, event-related potential (ERP) is a special brain evoked potential that utilizes multiple stimuli or multiple kinds of stimuli to induce brain potential by intentionally giving stimulation a special psychological meaning. For example, if the subject wants to quickly determine whether the pattern appearing on the screen is a target (such as red) or a non-target (such as green), compared to the non-target, when the target appears, the electrode of the parietal lobe is recorded to a positivity potential (referred to as the P300 potential) 300 to 500 milliseconds after the stimulation occurs.. The P300 potential is thought to be related to the response process of the person making the decision and may be used to interpret the person's choice. The self-generating potential is a continuous electrical activity resulting from the spontaneous activity of the brain cell population that is extracted and recorded from the scalp or cortex. This electrical activity is not specifically related to sensory input. Motion-imagery EEG is an EEG model when one imagines a certain limb movement. Visual-evoked potential (VEP) is a specific activity produced by the nervous system to receive stable visual stimuli such as graphic or flash stimuli.

In S102, the test information is input into a machine learning-based information recognition model, and a recognition result output from the information recognition model is obtained.

In the present embodiment, the machine learning-based information recognition model may be established in advance, and the model may be recognized by supervised learning and/or unsupervised learning training information. The information recognition model is used to be input with test information and to output a corresponding recognition result.

Optionally, the recognition result includes a name of the test information and/or a class to which the test information belongs.

In this embodiment, when machine learning-based information recognition model is adopted, the test information of the EEG-based psychological test does not need to be manually edited and classified, and can be directly input in a large amount. After the automatic recognition of the information recognition model, the content of the test information, or the class to which the test information belongs, or both the content of the test information and the class to which it belongs, can be obtained.

As an embodiment of the present disclosure, the test information is a test image, and the information recognition model is a first image recognition model trained by labeled image samples, where the first image recognition model is configured to recognize a name of an object in the test image or a class to which the object belongs.

In this embodiment, the first image recognition model may be established in advance, and the first image recognition model is trained by the training data set consisting of the labeled image samples. The test information can be multiple test images. After the test image is input into the first image recognition model, the first image recognition model outputs the recognized name of the object in the test image or the recognized class to which the object belongs.

For example, in finding a stimulus source scene that affects calmness, the image sample may be an image containing an object, the name of the object in the image sample is used as a label of the image sample, and the first image recognition model is trained. The trained first image recognition model can recognize the name of the object in the test image. In the psychological test scenario for drug abuser under detoxification, for the image sample, images such as drug, a needle, a drug-using environment, etc. are used as positive examples, and other images unrelated to drug are used as negative examples. The first image recognition model is trained, and the trained first image recognition model can recognize the class to which the object in the test image belongs, where the class to which the object belongs may include the first class related to drug use and the second class not related to drug use.

As an embodiment of the present disclosure, the test information is a test image, the information recognition model is a second image recognition model trained by the unlabeled image samples, and the second image recognition model is used to recognize the class to which the object in the test image belongs.

In this embodiment, the second image recognition model may be established in advance, and the second image recognition model is trained by the training data set consisting of the unlabeled image samples. The test information may be multiple test images. After the test image is input to the second image recognition model, the second image recognition model outputs the recognized class to which the object in the test image belongs.

For example, in a scene in which children's intelligence is tested, the image sample may be an image containing regular graphics (such as circles, squares, etc.) and an image containing irregular graphics. The image samples are unlabeled. The second image recognition model may be established and trained by clustering unsupervised learning methods. The trained first image recognition model can identify the class to which the graphic in the test image belongs. The class to which the graphic belongs may include a regular category and an irregular category.

In S103, a psychological test result is determined according to the EEG signal and the recognition result of the test information corresponding to the EEG signal in time.

In this embodiment, the EEG signal generated when the subject feels certain test information is the EEG signal corresponding to the test information, and the test information has a corresponding relationship with the EEG signal. The EEG signal and the test information corresponding to each other in time may be obtained, the recognition result corresponding to the test information is obtained, and the psychological test analysis is performed according to the EEG signal and the recognition result of the corresponding test information to determine the result of the psychological test.

For example, a play time identifier indicating the play time of the test information may be set for each test information, and a generation time identifier indicating the generation time of the signal is set for each EEG signal, and the correspondence between the test information and the EEG signal is determined according to the play time identifier of the test information and the generation time identifier of the EEG signal.

In this embodiment of the present disclosure, by automatically recognizing the test information by using a machine learning-based information recognition model, and then determining the psychological test result according to the EEG signal of the subject and the recognition result of the test information corresponding to the EEG signal in time, the test information can be recognized by using the information recognition model and there is no need to edit and collate the test information, thereby the test process can be simplified, the test efficiency can be improved, and the adoption range and data amount of the test information can be enlarged. Thus, the applicability of the psychological test scenario can be enhanced, the test result can be improved and psychological test can be made more convenient and practical.

It should be noted that the computer programs corresponding to the two steps S102 and S103 may be executed in the same terminal device, or may be executed in two different terminal devices or in multiple terminal devices. For example, the computer program corresponding to the two steps S102 and S103 may be executed in a user terminal such as a mobile terminal or the like; or the computer program corresponding to the step S102 is executed in the user terminal such as a mobile terminal or the like, and the computer program corresponding to the step S103 is executed in the server, which is not limited herein.

As an embodiment of the present disclosure, S103 may include:

determining a psychological test result according to the EEG signal, the recognition result of the test information corresponding to the EEG signal in time, and a preset rule; or

obtaining a psychological test result by inputting the EEG signal and the recognition result of the test information corresponding to the EEG signal in time into a machine learning-based psychological test model.

In this embodiment, the preset rule is an analysis rule of the psychological test set according to the actual application scene, for example, the analysis rules for the psychological test scene for drug abuser under detoxification, the analysis rules for finding a scene of the stimulus source that affects calmness, and the analysis rules for the scene of testing child's intelligence, which are not limited herein. The machine learning-based psychological test model is a model for automatically performing psychological test analysis, and the psychological test model adopted may be determined according to the actual application scene, which is not limited herein. In this embodiment the psychological test result may be determined based on the rules, and may be determined based on machine learning.

As an embodiment of the present disclosure, as shown in FIG. 2, the above-described method may further include:

In S201, a special EEG signal in the EEG signal is recognized according to a preset threshold.

In S202, test information corresponding to the special EEG signal is added into the training data set of the information recognition model, and the information recognition model is re-trained.

In this embodiment, the special EEG signal is an EEG signal corresponding to test information that has a large influence on the amplitude and latency of the frontal lobe of the subject, that is, an EEG signal generated when the subject feels the test information. The amplitude or frequency of these EEG signals is different from ordinary EEG signals. Therefore, special EEG signals in EEG signals can be recognized according to a preset threshold. For example, EEG signals with amplitude greater than a preset threshold may be determined as special EEG signals. The setting of the preset threshold may be determined according to the actual application scenario, which is not limited herein.

Since the test information corresponding to the special EEG signal has a great influence on the psychological test result, it can be determined whether the test information corresponding to the special EEG signal is in the training data set of the information recognition model, and if the test information corresponding to the special EEG signal is not in the training data set of the information identification model, the test information corresponding to the special EEG signal is added to the training data set of the information recognition model, and the information identification model is re-trained, thus the accuracy of the EEG psychological test can be improved.

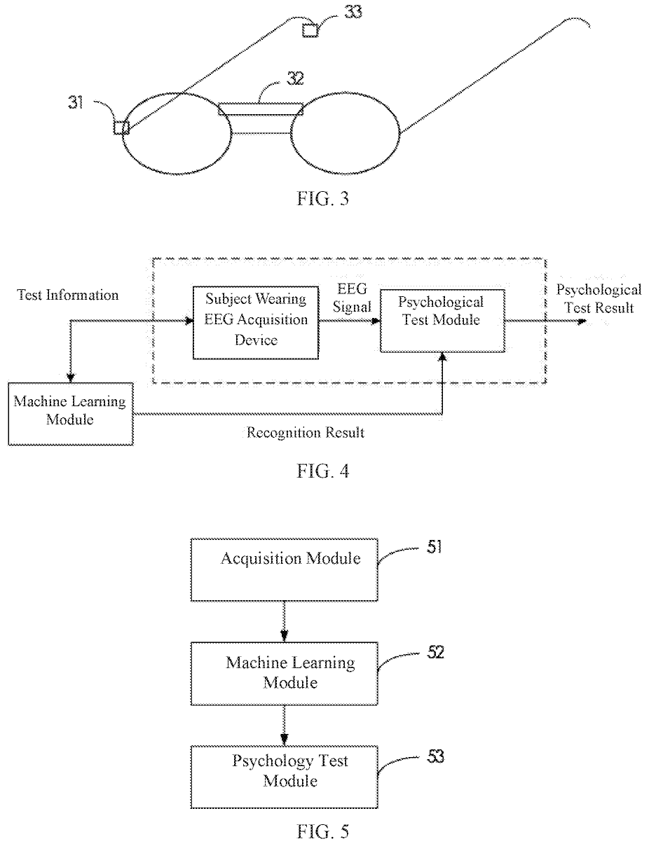

As an embodiment of the present disclosure, as shown in FIG. 3, the EEG acquisition device is a pair of glasses with a camera 31. The glasses is provided with a first electrode 32 at a nose bridge position thereof and a second electrode 33 at a position of a leg thereof adjacent to a wear's ear contact region.

In this embodiment, when the test information is visual information such as images, videos, characters, etc., the camera 31 may be used to collect test information. The setting position of the camera 31 on the glasses may be determined according to actual conditions, which is not limited herein. The first electrode 32 and the second electrode 33 are used to collect an EEG signal of the subject. The EEG acquisition device may send the collected test information and the EEG signal to the terminal device for data processing so as to realize psychological test. By using the glasses-type EEG acquisition device with camera, the test information and the EEG signal can be simultaneously collected, thereby improving the convenience of EEG-based psychological test, and enabling EEG acquisition device to adapt to more application scenarios.

As an embodiment of the present disclosure, in an application scenario in which a stimulus source that affects calmness is sought, an image recognition model may be established by a machine learning module. The image recognition model is created using a collection of labelled image samples (e.g., Imagenet). The image recognition model can output the name of an object in the test information (image or video) as a tag. The EEG acquisition device is a smart glasses with electrodes for measuring EEG signals. The test information is input to the machine learning module of the mobile phone by the smart glasses, and the machine learning module outputs the recognition result of the test information to the psychological test module. The EEG acquisition device outputs the EEG signal to the psychological test module on the mobile phone. The psychological test module outputs the test result according to the EEG signal and the recognition result corresponding to each other in time. Specifically, the resting state potential of the EEG signal may be regarded as a calm state, and the ERP signal or the VEP signal may be regarded as an unquiet state. The image label corresponding to the unquiet state is the stimulus source.

As an embodiment of the present disclosure, in an application scenario in which whether a successful detoxification is detected, a machine learning model may be established by a machine learning module. The machine learning model is created by using a collection of labeled images. For example pictures or texts of drugs, needles, and drug use environments are used as positive examples, and other pictures or texts that are not related to drug use are used as negative examples. The machine learning model may detect whether the input image and text are drug related or unrelated. The EEG acquisition device is a common device for measuring EEG signals, such as a headband. The test information comes from the collection of the common computing device, such as a computer. The machine learning module is also in the computer, and the test information is directly input into the machine learning module. The EEG acquisition device outputs the EEG signal to the computer or transmits it to the mobile device via Bluetooth, and the mobile device transmits the EEG signal to the computer by using WIFI communication. The machine learning module outputs the recognition result whether the data is drug related to unrelated to the psychological test module. The psychological test module outputs the test result according to the EEG signal and the recognition result corresponding to each other in time. Specifically, P300 and N200 of the EEG signal may be used as indicators. If the amplitude and latency of the frontal lobe vary greatly, the subject is not detoxified. In particular, if some of the recognition results have a particularly large impact on the amplitude and latency of the frontal lobe, but are not the training data, these recognition results may be considered to be added into the training set to re-train the machine learning model.

As an embodiment of the present disclosure, in an application scenario for detecting the development of children's intelligence, a machine learning module establishes a machine learning model. The machine learning model is established by using a collection of unlabeled images, such as regular graphics (circles, squares, etc.) and irregular graphics, which may be automatically classified by the distances between the center points of the graphics and the edges. The EEG acquisition device is a common devices for measuring EEG signals, such as a headband. The test information comes from the collection of a common computing device, such as a computer. The machine learning module is also in the computer, and the test information is directly input into the machine learning module. The EEG acquisition device outputs an EEG signal to the computer or transmits it to the mobile device via Bluetooth, and the mobile device transmits the EEG signal to the computer by using WWI communication. The machine learning module outputs the recognition result whether the graphics in the test information are regular or irregular to the psychological test module. The psychological test module outputs the test result according to the EEG signal and the recognition result corresponding to each other in time. Specifically, P300 and N200 of the EEG signal may be used as indicators. If the response to low-probability stimuli is significantly stronger than the response to high-probability stimuli while the magnitude of the two types of stimuli is significantly lower than the normal value, it may be determined that the subject's intellectual development is abnormal.

In the dashed box in FIG. 4 a traditional method of psychological test is shown. In the traditional method, the test information is input to the EEG acquisition device worn by the subject, the EEG acquisition device collects the EEG signal of the subject and sends the EEG signal to the psychological test module, and the psychological test module outputs the psychological test result. In the embodiment of the present disclosure, the test information is further input into the machine learning module, the machine learning module recognizes the test information and sends the recognition result of the test information to the psychological test module, and the psychological test module obtains the psychological test result according to the EEG signal and the corresponding recognition result. In the embodiment of the present disclosure, by using the machine learning model to extract the test information, and the scope of the test can be expanded. Because there is no need to artificially limit the class of test information, the psychological test can be more convenient and practical. The conventional method only has two steps in the dashed box. In the embodiment of the present disclosure the machine learning process for the test information is increased, thereby expanding the input range of test information and improving the efficiency of the psychological test.

In an embodiment of the present disclosure, a large amount of various data can be input, and for the scene of checking the stimulus source the scope of the monitoring can be effectively expanded, and for the scene of checking the mental state, the amount of data can be efficiently increased, and new situations that are not easily found in the fixed data input can be found. In an embodiment of the disclosure a new EEG acquisition device is proposed, in which the EEG detection electrodes and the camera are added on the glasses, which can make the psychological test more convenient.

In an embodiment of the present disclosure, by automatically recognizing the test information by using a machine learning-based information recognition model, and then determining the psychological test result according to the EEG signal of the subject and the recognition result of the test information corresponding to the EEG signal in time, the test information can be recognized by using the information recognition model and there is no need to edit and collate the test information, thereby the test process can be simplified, the test efficiency can be improved, and the adoption range and data amount of the test information can be enlarged. Thus, the applicability of the psychological test scenario can be enhanced, the test result can be improved and psychological test can be made more convenient and practical.

It should be understood that, values of serial numbers of the steps in the above embodiments don't mean the execution sequence of the steps, the execution sequence of the steps should be determined by its function and internal logics, and should not be construed as limiting the implementation process of the embodiments of the present application.

Corresponding to the EEG-based psychological test method described in the above embodiments, FIG. 5 is a schematic diagram of an EEG-based psychological test apparatus according to an embodiment of the present disclosure. For the convenience of explanation, only the parts related to the present embodiment are shown.

Referring to FIG. 5, the apparatus includes an acquisition module machine learning module 52, and a psychology test module 53.

The acquisition module 51 is configured to collect, by an EEG acquisition device, an EEG signal of a subject when test information is felt by the subject.

The machine learning module 52 is configured to input the test information into a machine learning-based information recognition model and to obtain a recognition result output from the information recognition model.

The psychological test module 53 is configured to determine a psychological test result according to the EEG signal and the recognition result of the test information corresponding to the EEG signal in time.

Optionally, the EEG signal comprises at least one selected from a group consisting of an event-related potential signal, a self-generating potential signal, a motion-imagery EEG signal and a visual-evoked potential signal.

Optionally, the recognition result includes a name of the test information and/or a class to which the test information belongs.

Optionally, the test information includes a test image, and the information recognition model includes a first image recognition model trained by labeled image samples, where the first image recognition model is configured to recognize a name of an object in the test image or a class to which the object belongs.

Optionally, the test information includes a test image, and the information recognition model includes a second image recognition model trained by unlabeled image samples, where the second image recognition model is configured to recognize a class to which an object in the test image belongs.

Optionally, the psychological test module 53 is configured to:

determine a psychological test result according to the EEG signal, the recognition result of the test information corresponding to the EEG signal in time, and a preset rule; or

obtain a psychological test result by inputting the EEG signal and the recognition result of the test information corresponding to the EEG signal in time into a machine learning-based psychological test model.

Optionally, the apparatus further includes a processing module configured to:

recognize a special EEG signal in EEG signals according to a preset threshold; and

add test information corresponding to the special EEG signal into a training data set of the information recognition model and re-train the information recognition model.

Optionally, the EEG acquisition device is a glasses with a camera, wherein the glasses is provided with a first electrode at a nose bridge position thereof and a second electrode at a position of a leg thereof adjacent to a wear's ear contact region.

In the embodiment of the present disclosure, by automatically recognizing the test information by using a machine learning-based information recognition model, and then determining the psychological test result according to the EEG signal of the subject and the recognition result of the test information corresponding to the EEG signal in time, the test information can be recognized by using the information recognition model and there is no need to edit and collate the test information, thereby the test process can be simplified, the test efficiency can be improved, and the adoption range and data amount of the test information can be enlarged. Thus, the applicability of the psychological test scenario can be enhanced, the test result can be improved and psychological test can be made more convenient and practical.

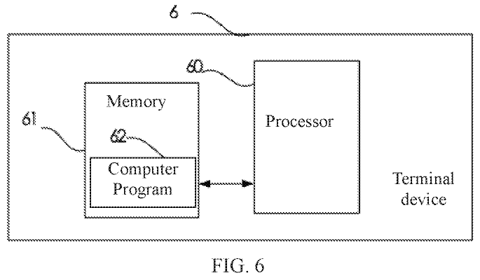

FIG. 6 is a schematic diagram of a terminal device according to an embodiment of the present disclosure. As shown in FIG. 6, the terminal device 6 of this embodiment includes a processor 60, a memory 61, and a computer program 62, such as a program, stored in the memory 61 and operable in the processor 60. The processor 60 is configured to execute the computer program 62 to implement the steps in each method embodiment described above, such as steps 101 to 103 shown in FIG. 1. Alternatively, the processor 60 is configured to execute the computer program 62 to implement the functions of the modules/units in each device embodiment described above, such as the functions of the modules 51 to 53 shown in FIG. 5.

Exemplarily, the computer program 62 can be divided into one or a plurality of modules/units, the one or plurality of modules/units are stored in the memory 61, and executed by the processor 60 so as to implement the present application. The one or plurality of modules/units can be a series of computer program instruction segments that can accomplish particular functionalities, these instruction segments are used for describing an executive process of the computer program 62 in the terminal device 6.

The terminal device 6 may be a computing device such as a desktop computer, a laptop, a palmtop computer, and a cloud server. The terminal device may include, but is not limited to, the processor 60 and the memory 61. It will be understood by those skilled in the art that FIG. 6 is merely an example of the terminal device 6, does not constitute a limitation of the terminal device 6, may include more or less components than those illustrated, or may combine some components, or may include different components. For example, the terminal device may further include an input/output device, a network access device, a bus, a display, and the like.

The processor 60 may be CPU (Central Processing Unit), and may alternatively be other general purpose processor, DSP (Digital Signal Processor), ASIC (Application Specific Integrated Circuit), FGPA (Field-Programmable Gate Array), or some other programmable logic devices, discrete gate or transistor logic device, discrete hardware component, etc. The general purpose processor may be a microprocessor, or alternatively, the processor may alternatively be any conventional processor or the like.

The memory 61 may be an internal storage unit of the terminal device 6, such as a hard disk or an internal storage unit of the terminal device 6. The storage device 61 may alternatively be an external storage device of the terminal device 6, such as a plug-in hard disk, a SMC (Smart Media Card), a SD (Secure Digital) card, a FC (Flash Card) or the like, equipped on the terminal device 6. Further, the memory 61 may include both the internal storage unit and the external storage device of the terminal device 6. The memory 61 is configured to store the computer programs, and other procedures and data needed by the terminal device 6 for determining wellbore cross-sectional shape. The memory 61 can also be configured to store data that has been output or being ready to be output temporarily.

It can be clearly understood by the one of ordinary skill in the art that, for describing conveniently and concisely, dividing of the aforesaid various functional units, functional modules is described exemplarily merely, in an actual application, the aforesaid functions can be assigned to different functional units and functional modules to be accomplished, that is, an inner structure of a data synchronizing device is divided into functional units or modules so as to accomplish the whole or a part of functionalities described above. The various functional units, modules in the embodiments can be integrated into a processing unit, or each of the units exists independently and physically, or two or more than two of the units are integrated into a single unit. The aforesaid integrated unit can by either actualized in the form of hardware or in the form of software functional units. In addition, specific names of the various functional units and modules are only used for distinguishing from each other conveniently, but not intended to limit the protection scope of the present application. Regarding a specific working process of the units and modules in the aforesaid device, reference can be made to a corresponding process in the aforesaid method embodiments, it is not repeatedly described herein.

In the aforesaid embodiments, the description of each of the embodiments is emphasized respectively, regarding a part of one embodiment which isn't described or disclosed in detail, please refer to relevant descriptions in some other embodiments.

The ordinarily skilled one in the art may aware that, the elements and algorithm steps of each of the examples described in connection with the embodiments disclosed herein can be implemented in electronic hardware, or in combination with computer software and electronic hardware. Whether these functions are implemented by hardware or software depends on the specific application and design constraints of the technical solution. The skilled people could use different methods to implement the described functions for each particular application, however, such implementations should not be considered as going beyond the scope of the present application.

It should be understood that, in the embodiments of the present application, the disclosed apparatus/terminal device and method could be implemented in other ways. For example, the apparatus/terminal device described above are merely illustrative; for example, the division of the modules or units is only a logical function division, and other division could be used in the actual implementation, for example, multiple units or components could be combined or integrated into another system, or some features can be ignored, or not performed. In another aspect, the coupling or direct coupling or communicating connection shown or discussed could be an indirect, or a communicating connection through some interfaces, devices or units, which could be electrical, mechanical, or otherwise.

The units described as separate components could or could not be physically separate, the components shown as units could or could not be physical units, which can be located in one place, or can be distributed to multiple network elements. Parts or all of the elements could be selected according to the actual needs to achieve the object of the present embodiment.

In addition, the various functional units in each of the embodiments of the present application can be integrated into a single processing unit, or exist individually and physically, or two or more than two units are integrated into a single unit. The aforesaid integrated unit can either be achieved by hardware, or be achieved in the form of software functional units.

If the integrated unit is achieved in the form of software functional units, and is sold or used as an independent product, it can be stored in a computer readable storage medium. Based on this understanding, a whole or part of flow process of implementing the method in the aforesaid embodiments of the present disclosure can also be accomplished by using computer program to instruct relevant hardware. When the computer program is executed by the processor, the steps in the various method embodiments described above can be implemented. Wherein, the computer program comprises computer program codes, which can be in the form of source code, object code, executable documents or some intermediate form, etc. The computer readable medium can include: any entity or device that can carry the computer program codes, recording medium, USB flash disk, mobile hard disk, hard disk, optical disk, computer storage device, ROM (Read-Only Memory), RAM (Random Access Memory), electrical carrier signal, telecommunication signal and software distribution medium, etc. It needs to be explained that, the contents contained in the computer readable medium can be added or reduced appropriately according to the requirement of legislation and patent practice in a judicial district, for example, in some judicial districts, according to legislation and patent practice, the computer readable medium does not include electrical carrier signal and telecommunication signal.

As stated above, the aforesaid embodiments are only intended to explain but not to limit the technical solutions of the present application. Although the present application has been explained in detail with reference to the above-described embodiments, it should be understood for the ordinary skilled one in the art that, the technical solutions described in each of the above-described embodiments can still be amended, or some technical features in the technical solutions can be replaced equivalently; these amendments or equivalent replacements, which won't make the essence of corresponding technical solution to be broken away from the spirit and the scope of the technical solution in various embodiments of the present application, should all be included in the protection scope of the present application.

What is claimed is:

1. An EEG-based psychological test method, comprising:
collecting, by an EEG acquisition device, an EEG signal of a subject when test information is felt by the subject;
inputting the test information into a machine learning-based information recognition model, and obtaining a recognition result output from the information recognition model; and
determining a psychological test result according to the EEG signal and the recognition result of the test information corresponding to the EEG signal in time.

2. The EEG-based psychological test method according to claim 1, wherein the EEG signal comprises at least one selected from a group consisting of an event-related potential signal, a self-generating potential signal, a motion-imagery EEG signal and a visual-evoked potential signal.

3. The EEG-based psychological test method according to claim 1, wherein the recognition result includes a name of the test information and/or a class to which the test information belongs.

4. The EEG-based psychological test method according to claim 1, wherein the test information includes a test image, and the information recognition model includes a first image recognition model trained by labeled image samples, wherein the first image recognition model is configured to recognize a name of an object in the test image or a class to which the object belongs.

5. The EEG-based psychological test method according to claim 1, wherein the test information includes a test image, and the information recognition model includes a second image recognition model trained by unlabeled image samples, wherein the second image recognition model is configured to recognize a class to which an object in the test image belongs.

6. The EEG-based psychological test method according to claim 1, wherein the step of determining a psychological test result according to the EEG signal and the recognition result of the test information corresponding to the EEG signal in time comprises:
determining a psychological test result according to the EEG signal, the recognition result of the test information corresponding to the EEG signal in time, and a preset rule; or
obtaining a psychological test result by inputting the EEG signal and the recognition result of the test information corresponding to the EEG signal in time into a machine learning-based psychological test model.

7. The EEG-based psychological test method according to claim 1, further comprising:
recognizing a special EEG signal in EEG signals according to a preset threshold; and
adding test information corresponding to the special EEG signal into a training data set of the information recognition model and re-training the information recognition model.

8. The EEG-based psychological test method according to claim 1, wherein the EEG acquisition device is a glasses with a camera, wherein the glasses is provided with a first electrode at a nose bridge position thereof and a second electrode at a position of a leg thereof adjacent to a wear's ear contact region.

9. A terminal device comprising a memory, a processor, and a computer program stored in the memory and operable in the processor, wherein the processor is configured to execute the computer program to implement steps of the method according to claim 1.

10. A computer readable storage medium with a computer program stored therein, wherein when the computer program is executed by a processor, steps of the method of claim 1 are implemented.
